(19) United States
(12) Patent Application Publication
(10) Pub. No.: US 2011/0038937 A1
(43) Pub. Date: Feb. 17, 2011

(54) Methods for delivering siRNA via Ionthophoresis

(75) Inventors: William Schubert, Chelmsford, MA (US); Peyman Moslemy, Mansfield, MA (US); Mike Patane, Andover, MA (US); Phil Isom, Westboro, MA (US)

Correspondence Address: Pierce Atwood LLP, 160 Federal Street, 10th Floor, Boston, MA, 02110, US

(73) Assignee: EyeGate Pharma S.A.S.

(21) Appl. No.: 12/745,112

(22) PCT Filed: Dec. 5, 2008
(86) PCT No.: PCT/US08/85709

§ 371 Date: Oct. 27, 2010

Related U.S. Application Data
(60) Provisional application No. 61/005,635, filed on Dec. 5, 2007.
(60) Provisional application No. 61/047,972, filed on Apr. 25, 2008.

(51) Int. Cl. A61K 9/14; A61K 31/7105; A61P 27/02; A61N 1/30

(52) U.S. Cl. 424/489; 514/44 A; 604/20; 977/773; 977/916

(57) ABSTRACT

Disclosed herein are formulations of siRNA suitable for delivery by ocular iontophoresis, devices for iontophoretic delivery of siRNA and methods of use thereof.

### RELATED APPLICATION

This application claims the benefit of U.S. provisional application 61/005,635, filed on Dec. 5, 2007, the entire contents of which are herein incorporated by reference.

### BACKGROUND

Oligonucleotides have been employed to treat various ocular diseases. Systemic, topical and injected formulations are employed for a variety of ophthalmic conditions. In particular, topical applications account for the widest use of non-invasively delivered oligonucleotides for ocular disorders. This approach, however, suffers from low bioavailability and, thus, limited efficacy.

Small interfering RNAs (siRNAs) are a class of double-stranded RNA oligonucleotides that have been used for treating various eye diseases. Ocular formulations are used that allow for diffusion of siRNA across an ocular membrane, however, such topical formulations suffer from slow, inadequate and uneven uptake. Because current ocular delivery methods achieve low ocular exposures, frequent applications are required and compliance issues are significant.

### SUMMARY OF THE INVENTION

The present invention relates to siRNA formulations and methods of use to maximize drug delivery and patient safety. The present invention pertains to formulations of siRNA suited for ocular iontophoresis. These novel formulations can be used to treat a variety of ocular disorders. The formulations are capable of being used with different iontophoretic doses (e.g., current levels and application times). These solutions can, for example: (1) be appropriately buffered to manage initial and terminal pHs, (2) be stabilized to manage shelf life (chemical stability), and/or (3) include other excipients that modulate osmolarity. Furthermore, the siRNA solutions are carefully crafted to minimize the presence of competing ions.

These unique dosage forms can address a variety of therapeutic needs. Ocular iontophoresis is a novel, non-invasive, out-patient approach for delivering an effective amount of siRNA into ocular tissues. This non-invasive approach leads to results comparable to or better than those achieved with ocular injections.

Topical siRNA applications involving ocular iontophoresis have not been described. Based on commercially available columbic-controlled iontophoresis for topical applications to the skin of a variety of therapeutics, it is clear that even well-understood pharmaceuticals require customized formulations for iontophoresis. These alterations maximize dosing effectiveness, improve the safety and manage commercial challenges. The known technical formulation challenges presented by dermatological applications may translate in to ocular delivery. Ocular iontophoresis, however, presents additional formulation needs. Thus, developing novel formulations that are ideally suited for ocular iontophoretic delivery of siRNA is required. Developing siRNA suitable for non-invasive local ocular delivery will significantly expand treatment options for ophthalmologists.

One embodiment is directed to a method of delivering therapeutically relevant oligonucleotides, small interfering RNA (siRNA), into the eye of a subject by transscleral iontophoresis, the method comprising the following steps: a. preparation of an ocular iontophoresis device containing an aqueous composition of oligonucleotide; b. placement of the device, connected to an electrical direct current generator, on the center of the eyeball surface such that the application surface is at least partly limited by an outer line concave towards the optical axis of the eyeball, and wherein the outer wall of the device extends from the outer line outwardly with respect to the optical axis; and c. administration of the oligonucleotide to the eye of the subject by performing iontophoresis, thereby delivering the oligonucleotide into the eye.

One embodiment is directed to a method of delivering an effective amount of siRNA via transscleral iontophoresis into the eye of a subject, comprising: a) placing a device on the center of the eyeball surface of the subject such that an application so surface is formed between the device and the eyeball, wherein the device comprises a reservoir containing an aqueous solution comprising one or more siRNA molecules or formulations thereof, and wherein the device is connected to an electrical generator; and b) administering the siRNA to the eye of the subject by performing iontophoresis, thereby delivering the siRNA into the eye. In a particular embodiment, the application of the device to the surface of the eyeball is at least partly limited by an outer line concave towards the optical axis of the eyeball, and wherein the outer wall of the device extends from the outer line outwardly with respect to the optical axis. In a particular embodiment, the siRNA is between about 15 and about 30 nucleotides in length. In a particular embodiment, the siRNA is between about 21 and about 23 nucleotides in length. In a particular embodiment, the reservoir contains a therapeutic composition comprising at least one oligonucleotide compound formulated in an aqueous solution suitable for ocular iontophoresis. In a particular embodiment, the therapeutic composition comprises at least agent selected from the group consisting of: a buffering agent, an osmotic agent, a permeation enhancer, a chelant, an antioxidant and an antimicrobial preservative. In a particular embodiment, the therapeutic composition is lyophilized prior to being reconstituted for iontophoresis application. In a particular embodiment, the reservoir contains an siRNA formulation in the form of a nanoparticle. In a particular embodiment, nanoparticle comprises at least agent selected from the group consisting of: a buffering agent, an osmotic agent, a permeation enhancer, a chelant, an antioxidant and an antimicrobial preservative. In a particular embodiment, the nanoparticle has a diameter between about 20 nm and about 400 nm. In a particular embodiment, the nanoparticle has a hydrodynamic diameter between about 40 nm and about 200 nm. In a particular embodiment, the nanoparticle has a zeta potential between about +5 mV and about +100 mV. In a particular embodiment, the nanoparticle has a zeta potential between about +20 mV and about +80 mV. In a particular embodiment, the nanoparticle has a zeta potential between about −5 mV and about −100 mV. In a particular embodiment, the nanoparticle has a zeta potential between about −20 mV and about −80 mV. In a particular embodiment, the nanoparticle is delivered by an iontophoretic current between about +0.25 mA and about +10 mA. In a particular embodiment, the nanoparticle is delivered by an iontophoretic current between about +0.5 mA and about +5 mA. In a particular embodiment, the reservoir holds between about 50 μL to about 500 μL of the siRNA formulation. In a particular embodiment, the reservoir holds between about 150 μL to about 400 μL of the siRNA formulation. In a particular embodiment, the administration time is between about 1 minute and about 20 minutes. In a particular embodiment, the administration time is between about 2 minutes and about 10 minutes. In a particular embodiment, the administration time is between about 3 minutes and about 5 minutes. In a particular embodiment, the siRNA in solution is delivered by an iontophoretic current between about −0.25 mA and about −10 mA. In a particular embodiment, the siRNA in solution is delivered by an iontophoretic current between about −0.5 mA and about −5 mA. In a particular embodiment, administration of siRNA occurs in a single dose. In a particular embodiment, administration of siRNA occurs over multiple doses. In a particular embodiment, the oligonucleotide is delivered by injection prior to iontophoresis. In a particular embodiment, the method of injection is selected from the group consisting of: an intracameral injection, an intracorneal injection, a subconjunctival injection, a subtenon injection, a subretinal injection, an intravitreal injection and an injection into the anterior chamber. In a particular embodiment, the oligonucleotide is administered topically prior to iontophoresis. In a particular embodiment, the step of ocular iontophoresis is carried out prior to, during or after the step of administering oligonucleotide.

One embodiment is directed to a method for treating ocular diseases in a mammal, comprising administering an effective amount of siRNA by ocular iontophoresis.

One embodiment is directed to an siRNA formulation suitable for ocular iontophoretic delivery into the eye of a subject. the formulation comprises a nanoparticle composition comprising the siRNA.

One embodiment is directed to a device for delivering siRNA to the eye of a subject, comprising: a) a reservoir comprising at least one medium comprising a siRNA formulation, the reservoir extending along a surface intended to cover a portion of an eyeball; and b) an electrode associated with the reservoir, wherein when the reservoir is placed in contact with the eyeball, the electrode can supply an electric field directed through the medium and toward a surface of the eye, thereby causing the siRNA to migrate into the eye and thereby delivering the siRNA formulation through the surface of the eye through iontophoresis. In a particular embodiment, the reservoir comprises: a) a first container for receiving the at least one medium comprising the siRNA formulation; b) a second container for receiving an electrical conductive medium comprising electrical conductive elements; and c) a semi-permeable membrane positioned between the first and second containers, the semi-permeable membrane being permeable to electrical conductive elements and non-permeable to the active substances.

### BRIEF DESCRIPTION OF THE DRAWINGS

FIG. 1 is a schematic diagram of an ocular iontophoresis system for delivering oligonucleotides, e.g., siRNA molecules, to a desired ocular tissue.

FIGS. 2A and B are fluorescence microscopy images of the conjunctiva and sclera of rabbit eyes treated iontophoretically with single-stranded oligonucleotide (ss-oligo) at a concentration of 1 mg/mL (FIG. 2A) and effects of passive diffusion for the same duration (FIG. 2B). Animals were treated with 15 mA·min of iontophoretic current (FIG. 2A) or no current (FIG. 2B). Scale bar represents 25 microns and applies to both Panels A and B.

FIGS. 3A and 3B are the intensity profiles generated from the images seen in FIG. 2. FIG. 3A shows the intensity profile of the ss-oligo after iontophoretic treatment while FIG. 3B represents the distribution of the ss-oligo after five minutes of passive diffusion. These images show both higher intensity as well as broader distribution indicating that more ss-oligo penetrated into the tissue after iontophoretic treatment as compared to passive diffusion.

FIGS. 4A-C are fluorescence microscopy images of ss-oligo distribution after iontophoretic delivery (FIG. 4A) as well as passive diffusion (FIGS. 4B and 4C) These images show that the ss-oligo has been delivered to a greater area of the eye after iontophoretic treatment as compared to passive diffusion.

FIGS. 5A and 5B are fluorescence microscopy images of the retina of a rabbit after iontophoretic treatment. FIG. 5A shows the distribution of ss-oligo in all layers of the retina. FIG. 5B shows the auto-fluorescence observed in this region of the retina indicating the signal recorded in FIG. 5A is due to the presence of the ss-oligo. Red=Cy5 labeled ss-oligo, Blue=nucleus, Green=auto-fluorescent signal found within retinal tissue.

FIG. 6 shows ss-oligo detected in aqueous humor in animals treated with a −4 mA current (Lanes 5-8) while no ss-oligo could be detected in the aqueous humor of rabbits treated passively (Lanes 1-4). Lane 9 shows that a known amount of ss-oligo spiked into water is detected at the same size as the experimental samples supporting the claim that iontophoretic delivery of the ss-oligo does not affect the integrity of the molecule. Concentration: 1 mg/mL; Duration 5 min; Current was either 0 mA or −3.0 mA; Control is 1 ng/mL of single-stranded oligo.

FIGS. 7A-D are fluorescence microscopy images (FIGS. 7A and 7B) and intensity profiles (FIGS. 7C and 7D) of the conjunctiva and sclera of rabbit eyes treated iontophoretically with Cy5-labeled double-stranded VEGF siRNA (1 mg/mL) (FIGS. 7B and 7D) and eyes treated with no current (FIGS. 7A and 7C). Animals were treated with no current for 5 minutes or 20 mA·min of iontophoretic current (−4 mA for 5 minutes). Scale bar represents 25 microns and applies to FIGS. 7A and 7B.

FIGS. 8A-B are fluorescence microscopy images of the limbal regions of rabbit eyes after passive diffusion (FIG. 8A) or iontophoretic treatment (FIG. 8B) showing the increase in the area of siRNA delivery after iontophoretic treatment. FIG. 8C is a graph comparing the deference in the distribution of siRNA after passive diffusion and iontophoretic treatment. Scale bar represents 250 microns and applies to both Panel A and B.

FIGS. 9A and 9B are fluorescence microscopy images of the conjunctiva (FIG. 9A) and lamina propria (FIGS. 9A and 9B) of rabbit eyes treated iontophoretically with Cy5-labeled double-stranded VEGF siRNA (1 mg/mL). These images show extensive cellular uptake after iontophoretic treatment. Scale bar represents 10 microns and applies to both FIGS. 9A and 9B. Red=Cy5 labeled VEGF siRNA, Blue=nucleus.

FIG. 10 shows siRNA detected in aqueous humor in animals treated with a −4 mA current (Lanes 1-4) while no siRNA could be detected in the aqueous humor of rabbits treated passively (Lanes 5-8). Lane 11 shows that a known amount of siRNA spiked into aqueous humor is detected at the same size as the experimental samples supporting the claim that iontophoretic delivery of the siRNA does not affect the integrity of the molecule. Concentration: 1 mg/mL; Duration 10 min; Current was either −4.0 mA or 0 mA; Control Lanes 9, 10 and 11 are siRNA spiked into Aqueous Humor at 0.5, 1 and 5 ng/mL respectively.

### DETAILED DESCRIPTION OF THE INVENTION

Described herein are compositions and methods for delivering siRNAs to the eye of a subject. Delivery of siRNAs is useful, for example, to treat various diseases (e.g., glaucoma, diabetic retinopathy, proliferative vitreoretinopathy, age-related macular degeneration (AMD), dry AMD, wet AMD, dry eye, etc.). Embodiments described herein are directed to the unexpected discovery that an effective amount of siRNA can be delivered via ocular iontophoresis. Delivery allows, for example, for the down-regulation of one or more specific genes, which results, for example, in the treatment of a particular disease or disorder.

As used herein, the term “small interfering RNA” refers to a class of about 18-25 nucleotide-long double-stranded RNA molecules. The average length of standard siRNA molecules is 21 or 23 nt. siRNA plays a variety of roles in biology. The present invention uses the RNA interference (RNAi) role of siRNA to specifically down regulate gene expression for treating various ocular conditions. Although the mechanism of RNAi involves a double-stranded RNA molecule, single-stranded or partially double-stranded RNA molecules can be delivered to a desired tissue, whereupon the single-stranded or partially double-stranded RNA molecules are converted to a desired double-stranded RNA molecule that down-regulates target gene expression. As used herein, the term “subject” refers to an animal, in particular, a mammal, e.g., a human.

Ocular iontophoresis is a technique in ophthalmic therapy that can overcome practical limitations with conventional methods of drug delivery to both the anterior and posterior sections of the eye (Eljarrat-Binstock, E. and Domb, A., J. Control Release, 110:479-489, 2006). Iontophoresis is a non-invasive technique in which a weak electric current is applied to enhance penetration of an ionized drug or a charged drug carrier into a body tissue. Positively charged substances can be driven into the tissue by electro-repulsion at the anode while negatively charged substances are repelled from the cathode. The simplicity of the application, the reduction of adverse side effects, and the enhanced drug delivery to the targeted region have resulted in extensive clinical use of iontophoresis mainly in the transdermal field. Ocular iontophoresis has been investigated extensively for delivering different active compounds including antibiotics (Barza, M. et al., Ophthalmology, 93:133-139, 1986; Rootman, D. et al., Arch. Ophthalmol., 106:262-265, 1988; Yoshizumi, M. et al., J. Ocul. Pharmacol., 7:163-167, 1991; Frucht-Pery, J. et al., J. Ocul. Pharmacol. Ther., 15:251-256, 1999; Vollmer, D. et al. J. Ocul. Pharmacol. Ther., 18:549-558, 2002; Eljarrat-Binstock, E. et al., Invest. Ophthalmol. Vis. Sci., 45:2543-2548, 2004; Frucht-Pery, J. et al., Exp. Eye Res., 78:745-749, 2004), antivirals (Lam, T. et al., J. Ocul. Pharmacol., 10:571-575, 1994), corticosteroids (Behar-Cohen, F. et al., Exp. Eye Res., 65:533-545, 1997; Behar-Cohen, F. et al., Exp. Eye Res., 74:51-59, 2002; Eljarrat-Binstock, E. et al., J. Control Release, 106:386-390, 2005), chemotherapeutic agents (Kondo, M. and Araie, M., Invest. Ophthalmol. Vis. Sci., 30:583-585, 1989; Hayden, B. et al., Invest. Ophthalmol. Vis. Sci., 45:3644-3649, 2004; Eljarrat-Binstock, E, et al., Curr. Eye Res., 32:639-646, 2007; Eljarrat-Binstock, E, et al., Curr. Eye Res., 33:269-275, 2008), and oligonucleotides (Asahara, T. et al., Jpn. J. Ophthalmol., 45:31-39, 2001; Voigt, M, et al., Biochem. Biophys. Res. Commun., 295:336-341, 2002). The process of iontophoresis involves applying a current to an ionizable substance, for example a drug product, to increase its mobility across a surface. Three principle forces govern the flux caused by the current, with the primary force being electrochemical repulsion, which propels like charged species through surfaces (tissues).

When an electric current passes through an aqueous solution containing electrolytes and a charged material (for example, the active pharmaceutical ingredient or API, or a formulation comprising an API), several events occur: (1) the electrode generates ions, (2) the newly generated ions approach/collide with like charged particles (typically the drug being delivered), and (3) the electrorepulsion between the newly generated ions force the dissolved/suspended charged particles (the API) into and/or through the surface adjacent (tissue) to the electrode. Continuous application of electrical current drives the API significantly further into the tissues than is achieved with simple topical administration. The degree of iontophoresis is proportional to the applied current and the treatment time.

Iontophoresis occurs in water-based preparations, where ions can be readily generated by electrodes. Two types of electrodes can be used to produce ions: (1) inert electrodes and (2) active electrodes. Each type of electrode requires aqueous media containing electrolytes. Iontophoresis with an inert electrode is governed by the extent of water hydrolysis that an applied current can produce. The electrolysis reaction yields either hydroxide (cathodic) or hydronium (anodic) ions. Some formulations contain buffers, which can mitigate pH shifts caused by these ions. Certain buffers can introduce like-charged ions that can compete with the drug product, i.e., the cargo to be iontophoresed, e.g., siRNA, for ions generated electrolytically, which can decrease delivery of the drug product. The polarity of the drug delivery electrode is dependent on the chemical nature of the drug product, specifically its pKₐ(s)/isoelectric point and the initial dosing solution pH. It is primarily the electrochemical repulsion between the ions generated via electrolysis and the drug product's charge (or the charge of the composition comprising an active agent, e.g., a nanoparticle formulation) that drives the drug product into tissues. Iontophoresis, therefore, offers a significant advantage over topical drug application, in that it increases drug delivery. The rate of drug delivery can be adjusted by varying the applied current, as determined by one of skill in the art.

Devices useful for iontophoretic delivery include, for example, the EyeGate® II applicator and related technology. The use of the EyeGate® II applicator and technology results in the use of less drug when compared to other devices, resulting in a reduction of the cost per treatment. The compositions and methods described herein utilize the ability of the EyeGate® II applicator and related technology to deliver therapeutically-relevant oligonucleotides into and through ocular tissues intact allowing their subsequent function.

The compositions and methods described herein allow for enhanced cellular uptake of the oligonucleotides obtained as a result of the iontophoretic treatment with the EyeGate® II applicator and technology. Use of the EyeGate® II applicator and technology to deliver the oligonucleotide to ocular tissue increases the cell permeability to this molecule as compared to topical methods of delivery. In addition, particular compositions, e.g., specifically-engineered nanoparticles, allow for more effective delivery, e.g., by creating a desired charge-to-mass ratio, and uptake by the cells, e.g., by incorporating uptake factors on the surface of the nanoparticle.

Methods of using double-stranded RNA, e.g., siRNA, for the targeted inhibition of gene expression are known to one of skill in the art. One of skill in the art would know to design the siRNA molecule to be homologous to an endogenous gene to be down-regulated, e.g., a gene that is abnormally expressed to cause a disease state. Sequences are selected according to known base-pairing rules. Methods and compositions described herein are useful for delivering the siRNA molecules to particular ocular tissue(s), as delivery and uptake has otherwise proven to be ineffective. Inconsistent results from previous siRNA methods involved delivery and uptake, not efficacy of the siRNA molecule after delivery and uptake to a specific tissue. The methods described herein, therefore, enhance the delivery and uptake of siRNA molecules into a specific, desired tissue, wherein the siRNA function of the particular molecule allows for the down-regulation of a desired gene product, thereby effectively treating a disease associated with the gene product. An effective amount of a particular siRNA is sufficient to produce a clinically-relevant down-regulation of a particular gene, as determined by one of skill in the art. As used herein, the term “effective amount” refers a dosage of siRNA necessary to achieve a desired effect, e.g., the down-regulation of a specific gene target to the degree to which a desired effect is obtained. The term “effective amount” also refers to relief or reduction of one or more symptoms or clinical events associated with ocular disease.

For the purposes of the compositions and methods described herein, the siRNA is between about 15 to about 30 nucleotides in length, e.g., between 22 to 23 nucleotides in length. The siRNA molecule can be fully double-stranded, partially double-stranded, or single-stranded, as one of skill in the art would be able to generate molecules that either start out as double-stranded RNA molecules, or would be converted to double-stranded RNA molecules in vivo after uptake into a desired tissue or cell. It would be appreciated by one of skill in the art that, as the methods described herein rely on the physical properties of RNA or formulations comprising RNA generally, e.g., a charge-to-mass ratio, the methods and compositions are sequence independent, at least with regard to delivery and uptake (Brand, R. et al., J. Pharm. Sci., 49-52, 1998).

After delivery and uptake by a desired ocular tissue, the siRNA molecule effectively down-regulates the endogenous gene expression of the desired target gene. Particular examples of target genes include, but are not limited to, for example, beta adrenergic receptors 1 and/or 2; carbonic anhydrase II; cochlin; bone morphogen protein receptors 1/2; gremlin; angiotensin-converting-enzyme; angiotensin II type 1 receptor (AT1); angiotensinogen (ANG); renin; complement D; complement C3; complement C5; complement C5a; complement C5b; complement Factor H; VEGF; VEGF receptors (1, 2 or both); integrin αᵥ, β₃; PDGF receptor β; protein kinase C; c-JUN transcription factor; IL-1 alpha; IL-1 beta; TNFalpha; MMP; ICAM-1; insulin like growth factor-1; insulin like growth factor-1 receptor; growth hormone receptor GHr; integrins αᵥ β₅; TNFα; ICAM-1; MMP-10; MMP-2; MMP-9; etc.

The siRNA of the present invention can be encapsulated in the form of a nanoparticle. In certain embodiments, a specific uniform charge-to-mass ratio is achieved where an API is encapsulated in a nanoparticle, depending on the precise nature of the nanoparticle. Encapsulating an API in a nanoparticle also allows, for example, for increased residence time of the API, increased uptake into a particular cell, molecular targeting of the API to a particular target within a desired tissue or cell, increased stability of the API, and other advantageous properties associated with specific nanoparticles.

The siRNA formulation or composition can be contained, for example, in solution, e.g., a solution that serves to preserve the integrity of the formulation and/or serves as a suitable iontophoresis buffer. The solution can be optimized, for example, for the iontophoretic delivery of the oligonucleotide to ocular tissues while ensuring the stability of the oligonucleotide before and during the iontophoretic delivery using the EyeGate® II applicator and technology. The formulation and/or solution can also be designed for compatibility with the ocular tissue it will encounter.

The use of the EyeGate® II applicator and technology to deliver the oligonucleotide, or the oligonucleotide-loaded nanoparticles, can be further enhanced by modifying the applicator to ensure constant buffering of the solution as well as minimizing the volume of solution needed to successfully complete the iontophoretic treatment. These two objectives are completed by the addition of a buffering system to the applicator. The use of a buffering system in the applicator ensures the safety of the patient and maintain the integrity of the oligonucleotide during the iontophoretic treatment.

The addition of the membrane-shaped buffering system to the EyeGate® II applicator can also reduce the volume of the foam insert that serves as a reservoir for drug-containing solution. The foam insert is made of a rapidly swellable hydrophilic polyurethane based foam matrix shaped as a hollow cylinder with approximate dimensions of 6 mm (length)×14 mm (inside dia.)×17 mm (outside dia.). As a result, the overall volume of drug containing solution needed to hydrate the foam insert is reduced. For instance, incorporation of a 3 mm thick hydrogel/membrane buffer system can result in an overall reduction of drug containing solution by 50%, compared to the amount needed in a standard EyeGate® II applicator. Each 1 mm of the foam insert removed from the applicator corresponds to approximately 16% reduction in drug containing solution needed to fill the reservoir. As such, the amount of drug containing solution can be tailored to meet the specific needs of the individual treatment regimen.

The EyeGate® II applicator and technology can be used to deliver nanoparticle preparations of therapeutically-relevant oligonucleotides into and through ocular tissues. The nanoparticles can then release their payload (e.g., active agent, siRNA oligonucleotide) in a time- and/or rate-controlled fashion to deliver oligonucleotides in an intact state, thereby allowing their cellular uptake and subsequent function. Regardless of the oligonucleotide size, nucleotide composition and/or modifications to the oligonucleotide, the EyeGate® II applicator and technology does not affect the integrity of the oligonucleotide.

Pre-fabricated oligonucleotide-loaded nanoparticles can be used to deliver siRNA molecules to a desired ocular tissue via iontophoresis. Reviews of nanoparticles for ocular drug delivery are available (Zimmer, A. and Kreuter. J., Adv. Drug Delivery Reviews, 16:61-73, 1995; Amrite and Kompella, Nanoparticles for Ocular Drug Delivery, In: Nanoparticle Technology for Drug Delivery, Vol 159, Gupta and Kompella (eds.), 2006; Kothuri et al., Microparticles and Nanoparticles in Ocular Drug Delivery, In: Ophthalmic Drug Delivery Systems, Vol. 130, Ashim K. Mitra (ed.), 2nd edition, 2008).

Materials used in fabrication of nanoparticles for ocular delivery include, but are not limited to, polyalkylcyanoacrylates such as, for example, poly(ethylcyanoacrylate), poly(butylcyanoacrylate), poly(isobutylcyanoacrylate), poly(hexylcyanoacrylate), poly(hexadecyl cyanoacrylate), or copolymers of alkylcyanoakrylates and ethylene glycol; a group consisting of poly(DL-lactide), poly(L-lactide), poly(DL-lactide-co-glycolide), poly(ε-caprolactone), and poly(DL-lactide-co-ε-caprolactone); or a group consisting of Eudragit® polymers such as Eudragit® RL 100, Eudragit® RS 100, Eudragit® E 100, Eudragit® L 100, Eudragit® L 100-55, and Eudragit® S 100. The nanoparticles can be also fabricated from, for example, polyvinyl acetate phthalate, cellulose acetate phthalate, hydroxypropyl methylcellulose phthalate, or hydroxypropyl methylcellulose acetate succinate. The materials can include, for example, natural polysaccharides such as, for example, chitosan, alginate, or combinations thereof; complexes of alginate and poly(1-lysine); pegylated-chitosan; natural proteins such as albumin; lipids and phospholipids such as liposomes; or silicon. Other materials include, for example, polyethylene glycol, hyaluronic acid, poly(1-lysine), polyvinyl alcohol, polyvinyl pyrollidone, polyethyleneimine, polyacrylamide, poly(N-isopropylacrylamide).

### EXEMPLIFICATION

### Example 1

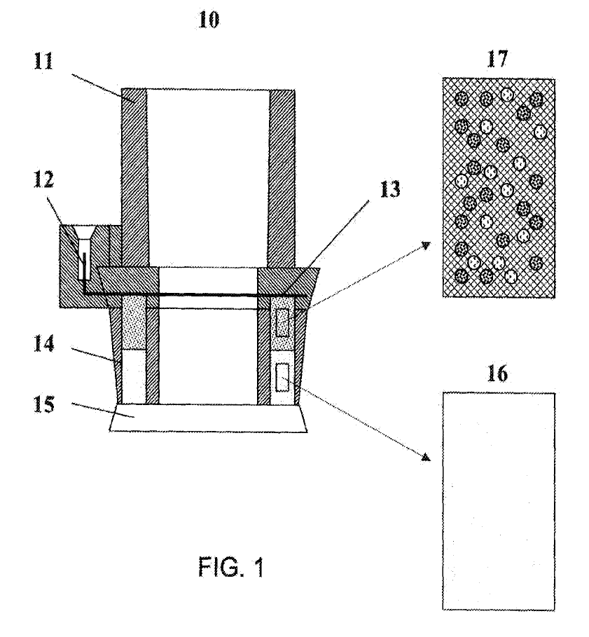

FIG. 1 illustrates the longitudinal cross-section of an ocular iontophoresis device, EyeGate® II applicator, consisting of a foam insert saturated with an oligonucleotide aqueous solution and a hydrogel matrix/membrane containing a buffer composition. The shapes, sizes, and relative positions of device elements in the drawing are not necessarily precise or drawn to scale. The particular shapes of the elements as drawn, are not intended to convey any information regarding the actual shape of the particular elements, and have been solely selected for ease of recognition in the drawings. The drug formulation reservoir consists of: (i) a foam insert saturated with a liquid preparation comprising one or more therapeutic oligonucleotide compounds, optionally a buffer composition, and optionally inactive ingredients pharmaceutically acceptable for ophthalmic delivery; and, optionally, (ii) a hydrogel matrix/membrane containing a buffer composition. At least one therapeutic compound is dissolved in the solution. The buffer composition is: (i) a plurality of ion exchange resin particles including cation and or anion exchange resins; (ii) a plurality of polymeric particles including cationic and or anionic particles; (iii) a cationic and or anionic polymer; (iv) a biological buffer; or (v) an inorganic buffer. Particles can have regular (e.g., round, spherical, cube, cylinder, fiber, and needle) or irregular shape. The applicator (10) consists of the following main elements:
11. a proximal part that provides rigid support for the device and a means to transfer drug formulation to the reservoir;
12. a source connector pin that provides a connection point between the current generator and the electrode;
13. an electrode that transfers the current to the formulation reservoir;
14. a reservoir that contains the drug formulation to be delivered;
15. a distal part, which is a soft plastic that interfaces with the eye; and
16. a therapeutic oligonucleotide compound dissolved in a liquid solution saturating the foam insert.

### Example 2

### In Vivo Delivery of Anti-VEGF siRNA

Female New Zealand white rabbits weighing approximately 3 kg each are housed at least three days prior to treatment in order to recover from shipping and to acclimate to the facility environmental conditions. The hair on the back of both ears is removed with a hair removal cream at least 24 hours prior to treatment. Animals are anesthetized 20 minutes prior to treatment with an intramuscular injection of ketamine (35 mg/kg) and xylazine (5 mg/kg). Once the animals are anesthetized return electrodes are placed on the bare skin of the ears (one patch per ear) and connected to the generator. Using a 1 mL syringe with a 27 gauge needle about 0.25 mL to about 0.50 mL of the siRNA containing solution is added to the foam insert in multiple sets of EyeGate® II applicators as needed. Each applicator is visually inspected to ensure complete hydration of the foam. Any air bubbles or unhydrated regions are mechanically removed. The EyeGate® II applicator is then connected to the generator and placed on the right eye after a drop of topical anesthetic is applied. The proper treatment is administered and the device is taken off of the eye. The animal is then turned over and the process repeated on the left eye. The remaining rabbits receive iontophoretic doses of siRNA each with a new applicator in a similar fashion.

Rabbits can receive a 4 mA current treatment lasting for 10 min in each eye (total iontophoretic dose of 40 mA·min) starting with the right eye. Immediately after the treatment of the left eye is completed for each animal, 1 mL of blood is removed and spun down to collect plasma samples. After the blood sample is taken, the animal is euthanized. All animals are euthanized with a 4 mL overdose of Euthasol injected intravenously into the marginal ear vein. Death is confirmed by the absence of a heart beat and lack of breathing. Once death is confirmed, the aqueous humor from each eye is removed using a 0.33 mL insulin syringe and placed in a DNAse and RNAse free tube and stored at −80° C. until analyzed. The eyes are then enucleated and dissected into its constituent components with each tissue type placed in separate DNAse and RNAse free tubes and stored at −80° C. until analyzed by mass spectrometry for quantitation and integrity determination.

### Example 3

### Transscleral Delivery of a 7.5 kDa Single-Stranded Oligonucleotide

Iontophoretic mobility of single stranded RNA molecules was examined in ocular tissue in vivo.

White New Zealand rabbits (˜3 kg) received a single dose of single-stranded RNA oligonucleotide at 1 mg/mL concentration using the EyeGate® II device with a current of 3 mA for 5 minutes, resulting in a total iontophoretic dose of 15 mA·min.

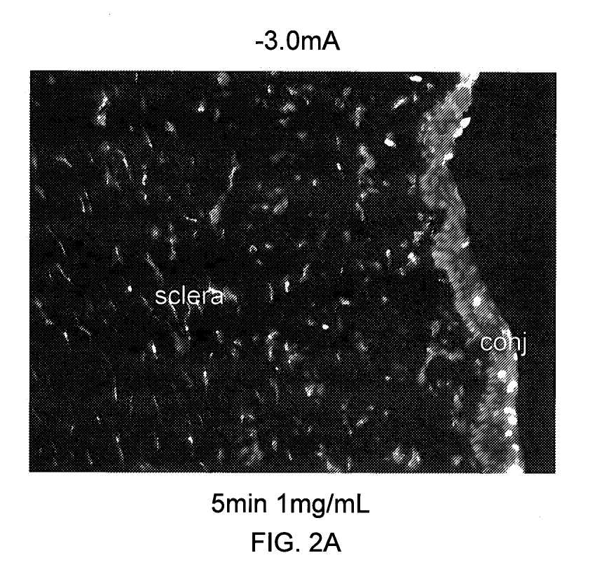

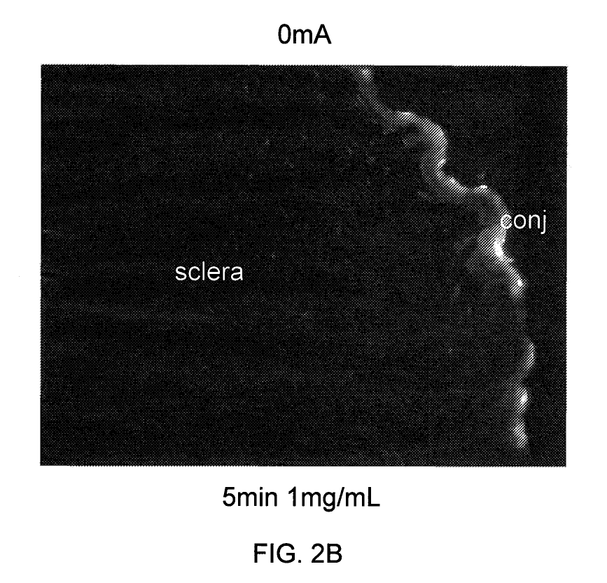

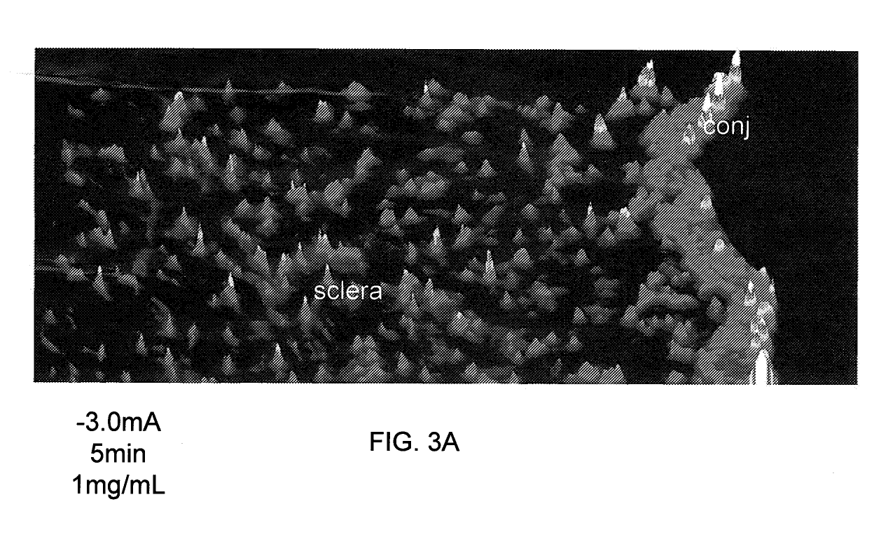

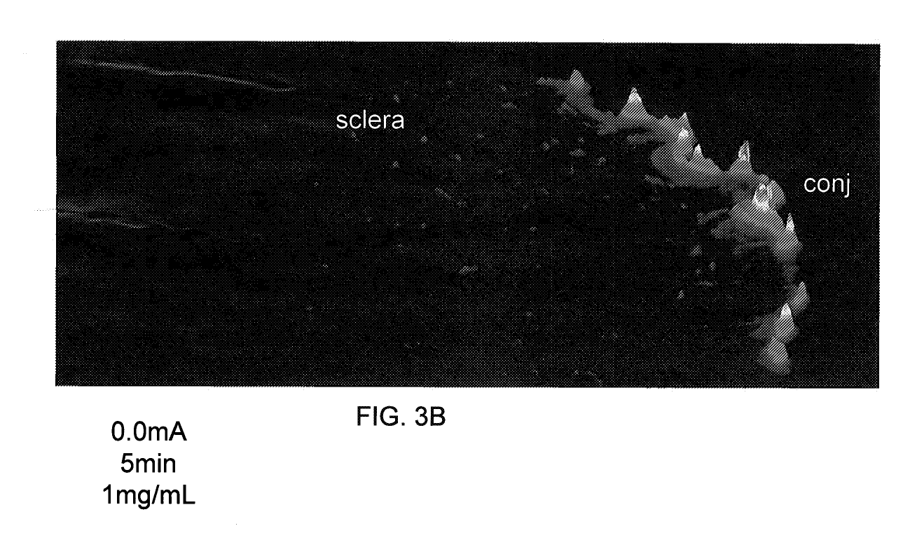

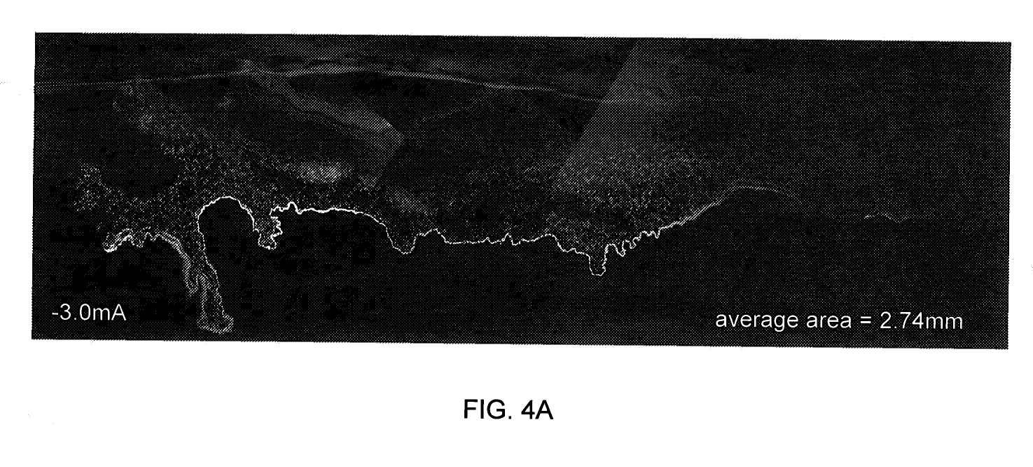

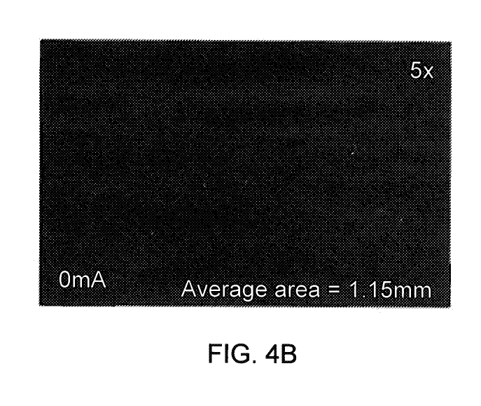

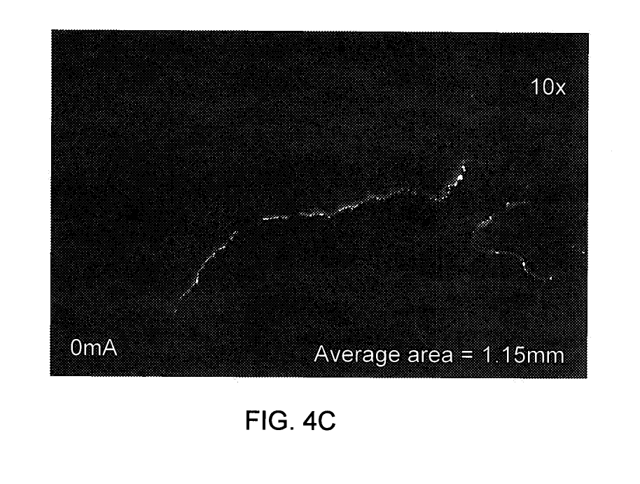

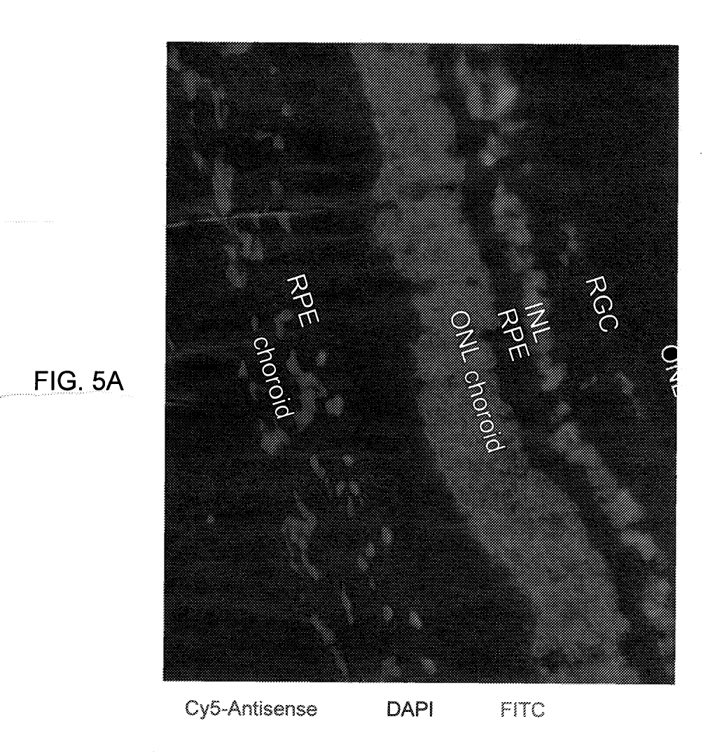

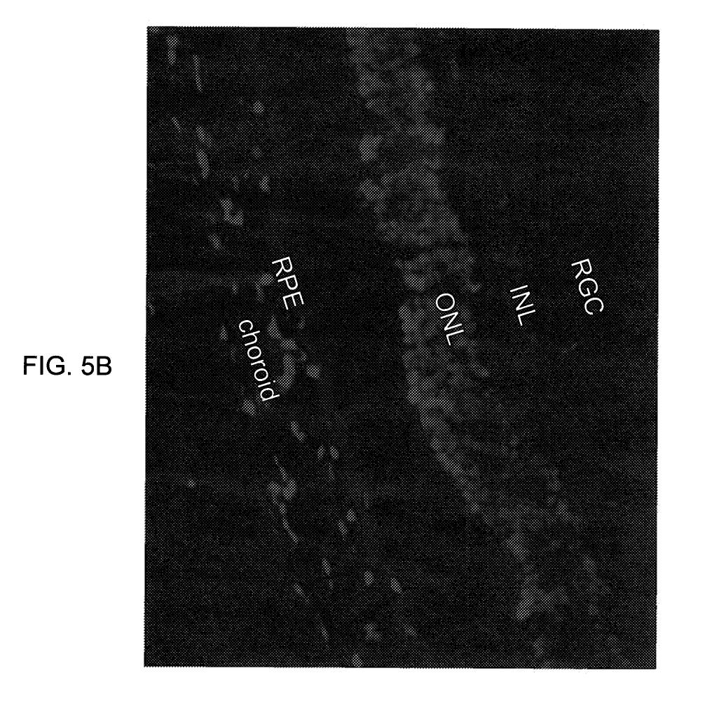

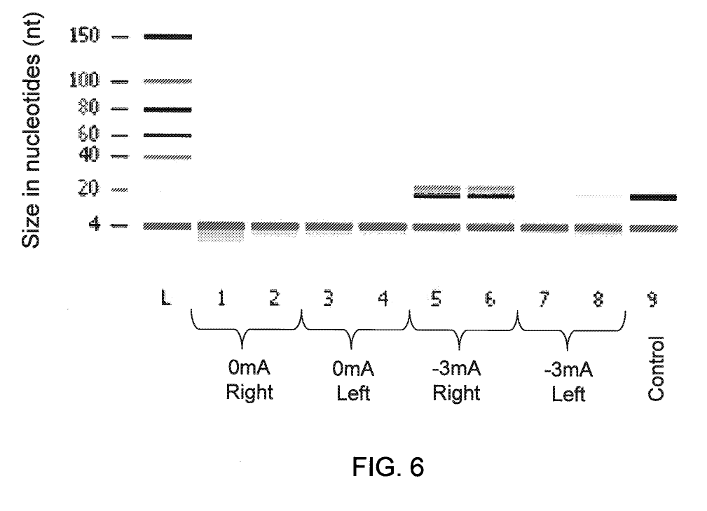

Iontophoresis of the single-stranded oligonucleotide into rabbit eyes using the EyeGate® II device increased the amount of oligonucleotide transported into the ocular tissues as compared to passive diffusion (FIGS. 2, 3, and 5). The iontophoretic treatment also increased the area to which the oligonucleotide was delivered as compared to passive diffusion (FIG. 4). The integrity of the oligonucleotide was also unaffected after the iontophoretic treatment (FIG. 6).

### Example 4

### Transscleral Delivery of a 15 kDa Double-Stranded siRNA

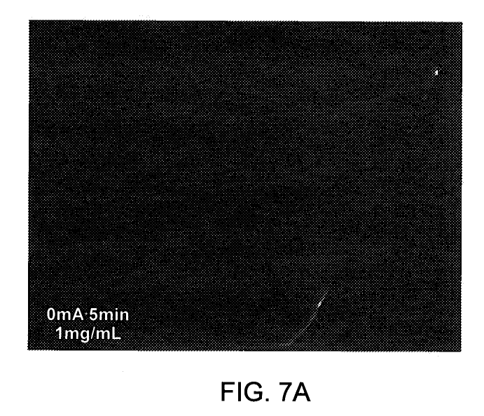

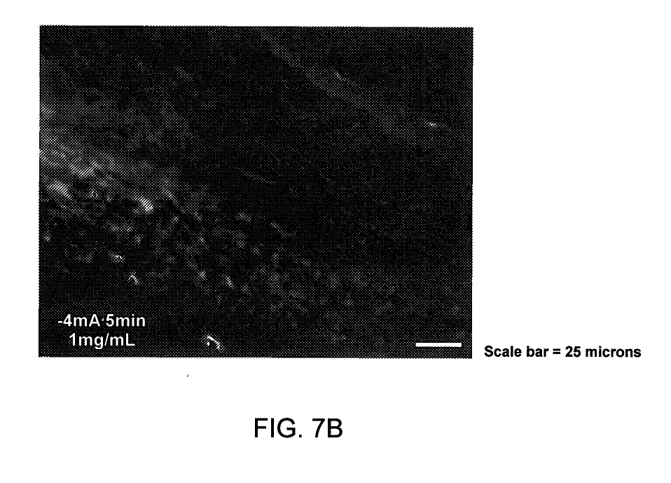

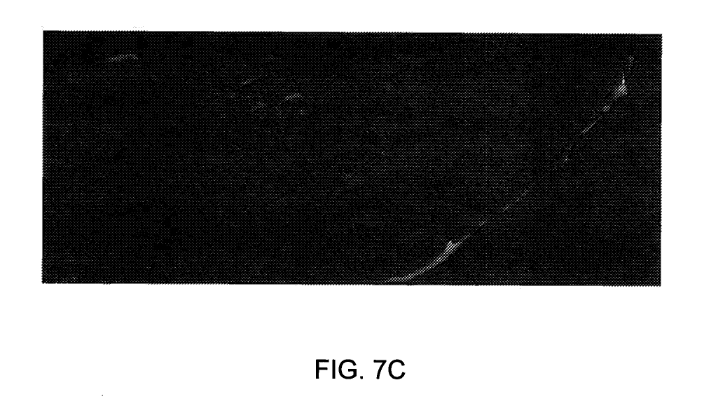

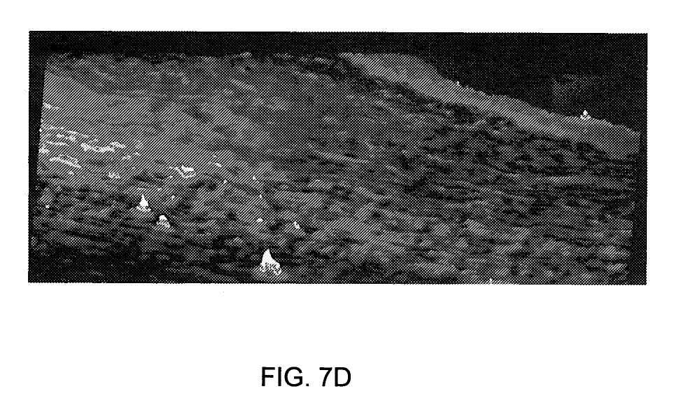

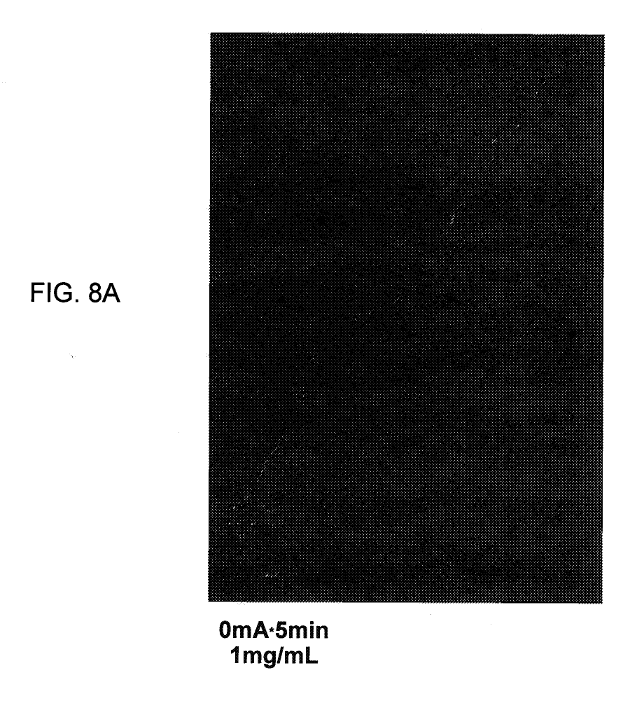

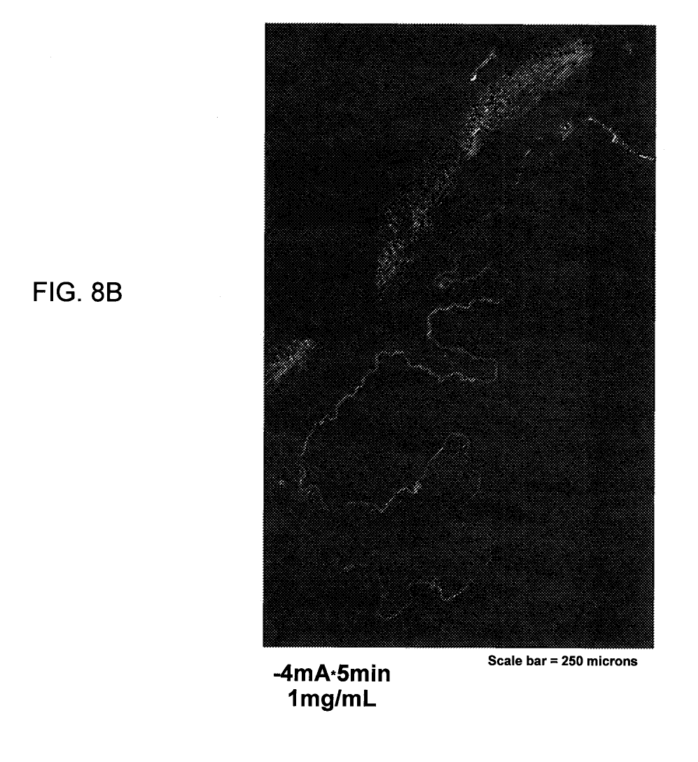

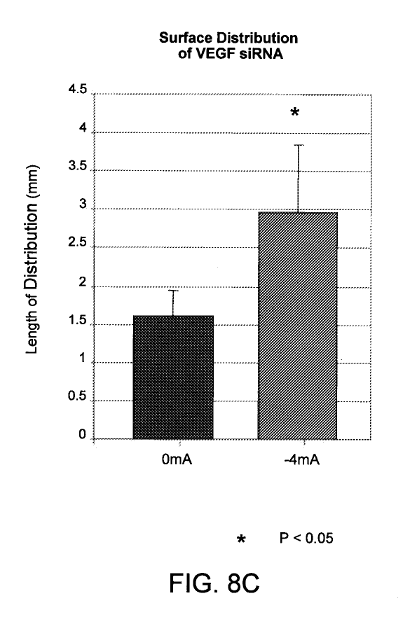

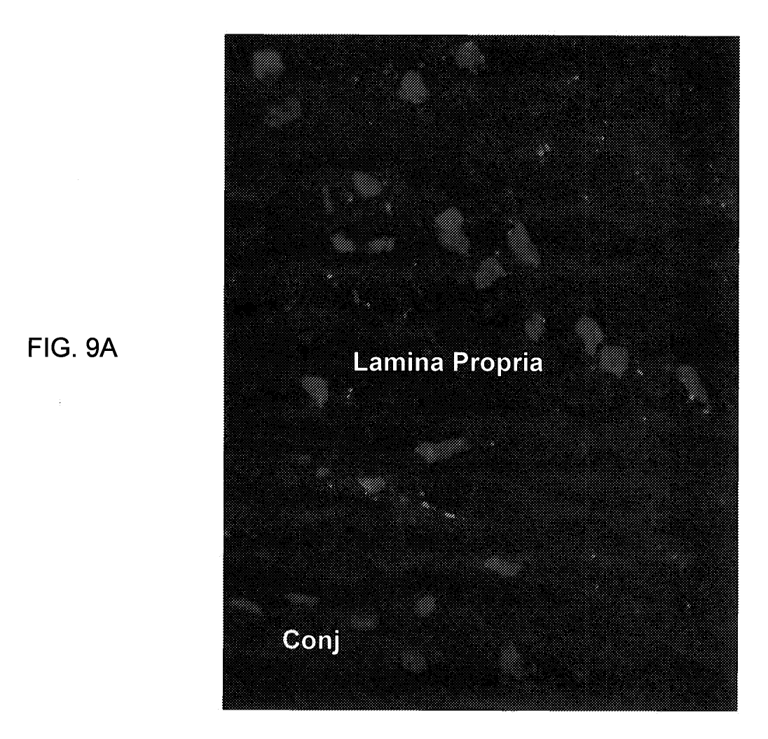

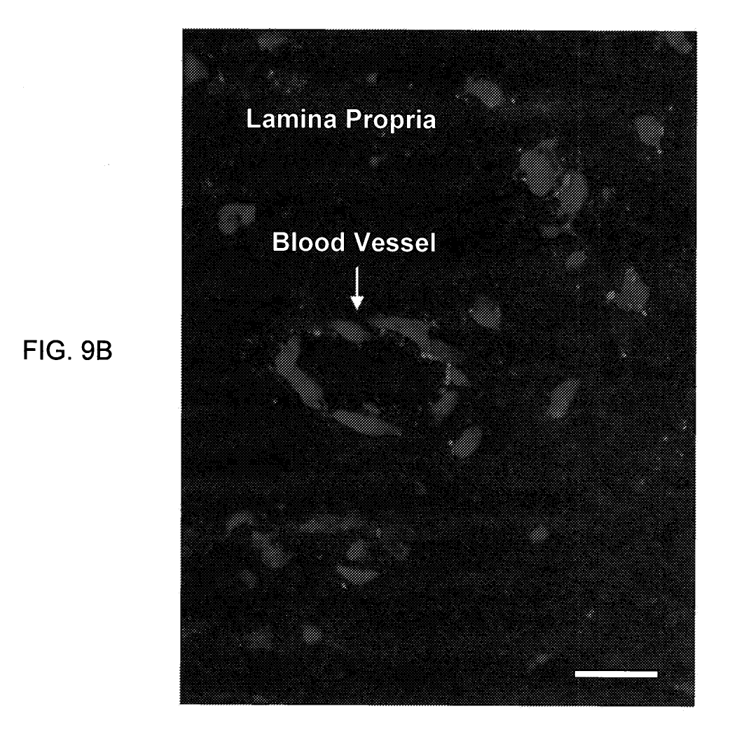

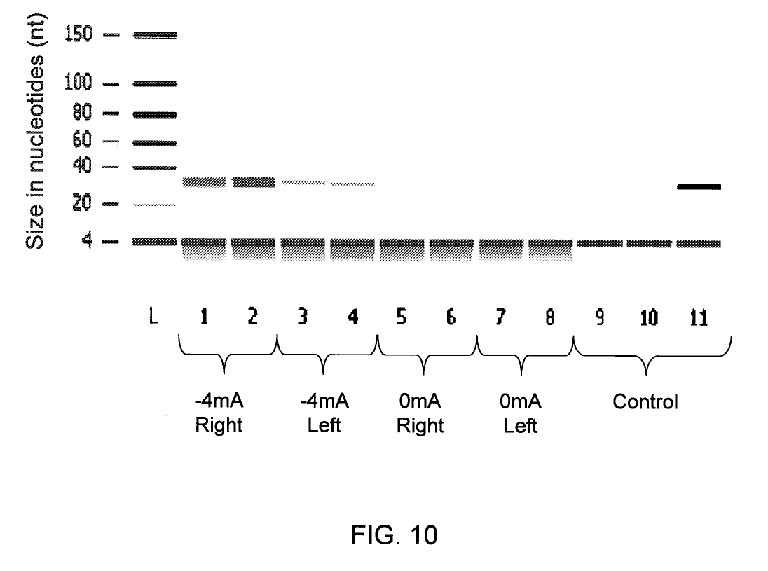

A 15 kDa double stranded Vascular Endothelial Growth Factor (VEGF) siRNA molecule effective in treating age related macular degeneration was tested. The anti-VEGF siRNA molecules (labeled with Cy5 for detection by fluorescence microscopy) were delivered in New Zealand rabbit eyes by iontophoresis using the EyeGate® II device (FIGS. 7-10). As seen with the single-stranded oligonucleotide, iontophoresis using the EyeGate® II device increased the amount of oligo delivered to the various ocular tissues as compared to passive diffusion (FIG. 7) as well as the overall area to which the siRNA was delivered to (FIG. 8). An iontophoretic treatment using the EyeGate® II device also resulted in an increase in the amount of cellular uptake of the anti-VEGF siRNA observed as compared to passive diffusion (FIG. 9). In addition, the integrity of the siRNA oligonucleotide was also unchanged after the iontophoretic treatment (FIG. 10).

Additional disease and gene targets are summarized and listed in Table 1.

TABLE 1

| | | Primary | Ocular tissue | Protein | RNA |
|---|---|---|---|---|---|
| Target | Mechanism of Action | indication | Distribution | expression | expression |
| Beta | The human trabecular meshwork | Glaucoma | ciliary body, | ciliary body, | |
| Adrenergic | and ciliary body, which express | | endothelial cells | endothelial cells | |
| receptor 1 | ADRβ1 and ADRβ2, control | | | | |
| and 2 | aqueous humor dynamics and | | | | |
| | blood flow | | | | |
| Carbonic | Carbonic anhydrase II in the | Glaucoma | Corneal | Corneal | |
| anhydrase II | ciliary processes of the eye | | endothelium, | endothelium, | |
| | regulates aqueous humor | | epithelium of | epithelium of | |
| | secretion, through its | | ciliary process and | ciliary process and | |
| | involvement In proton and | | lens, retinal Muller | lens, retinal Muller | |
| | bicarbonate transmembrane | | cells and some | cells and some | |
| | transport-facilitating the | | cones, choroidal | cones, choroidal | |
| | movement of other solutes | | and ciliary process | and ciliary process | |
| | across the membrane leading to | | endothelium | endothelium | |
| | acid-base homeostasis and fluid | | | | |
| | movement. | | | | |
| Cochlin | Increased deposition in the ECM | Glaucoma | Trabecular | ECM of the | Trabecular |
| | of the TM increases IOP by | | meshwork cells | Trabecular | meshwork |
| | altering AH flow dynamics. | | | meshwork | cells |
| | Increased deposition results in | | | | |
| | fibrillar collagen interaction | | | | |
| | resulting in collagen degradation | | | | |
| | and debris accumulation. | | | | |
| Bone | blocks BMP ligand binding and | Glaucoma | Trabecular | Trabecular | Trabecular |
| Morphogen | subsequent signaling | | cells/Optic nerve | cells/Optic nerve | cells/Optic |
| Protein | | | head Astrocytes | head Astrocytes | nerve head |
| Receptors | | | | | Astrocytes |
| 1/2 | | | | | |
| Gremlin | extracellular BMP antagonist | Glaucoma/ | Trabecular | Trabecular | Trabecular |
| | | Diabetic | cells/Optic nerve | cells/Optic nerve | cells/Optic |
| | | retinopathy/ | head | head | nerve head |
| | | Proliferative | Astrocytes/Retinal | Astrocytes/Retinal | Astrocytes |
| | | vitreo- | vasculature | vasculature | |
| | | retinopathy | | | |
| angiotensin- | Unknown-Decrease | Glaucoma/ | RPE/Choriod/ | | RPE/ |
| converting- | outflow: inhibition results in | AMD/ | Retina | | Choriod/ |
| enzyme | decreased formation of | Diabetic | | | Retina |
| | Angiotensin II (a more potent | retinopathy | | | |
| | vasoconstrictor than Angiotensin | | | | |
| | I) and decreased inactivation of | | | | |
| | bradykinin a vasodilator | | | | |
| angiotensin | The major pathogenic signaling | Glaucoma/ | ciliary body, | ciliary body, | endothelial |
| II type 1 | of angiotensin II is mediated by | AMD/ | endothelial cells | endothelial cells | cells |
| receptor | AT1-R (over expression of | Diabetic | | | |
| (AT1) | ICAM-1). AT1-R downstream | retinopathy | | | |
| | signaling leads to the activation | | | | |
| | of NF-κB, which plays a role in | | | | |
| | the regulation of gene | | | | |
| | expression of inflammation- | | | | |
| | related molecules including | | | | |
| | adhesion molecules, | | | | |
| | chemokines, and cytokines. | | | | |
| Angioten- | Precursor to Angiotensin II a | Glaucoma/ | RPE/Choriod/ | | RPE/Choriod/ |
| sinogen | potent vasoconstrictor | AMD/ | Retina | | Retina |
| (ANG) | | Diabetic | | | |
| | | retinopathy | | | |
| Renin | Enzyme that cleaves substrate | Glaucoma/ | RPE/Choriod/ | | RPE/Choriod/ |
| | angiotensinogen to form | AMD/ | Retina | | Retina |
| | Angiotensin I a precursor to | Diabetic | | | |
| | Angiotensinogen II a potent | retinopathy | | | |
| | vasoconstrictor | | | | |
| Complement D | Cleavage of C3-factor B complex | Dry AMD | | | |
| | by Factor D forms an alternative | | | | |
| | C3 convertase allowing cleavage | | | | |
| | of C5 resulting in C5a and C5b-9 | | | | |
| | pro-Inflammatory cleavage | | | | |
| | products | | | | |
| Complement | Initiation of the alternate pathway | Dry AMD | | | Glial cells |
| C3 | begins with the spontaneous | | | | |
| | conversion of C3 in serum to | | | | |
| | C3(H₂O). C3(H₂O) forms a | | | | |
| | complex with Mg₂ and factor B, | | | | |
| | which is susceptible to the | | | | |
| | enzymatic action of factor D, | | | | |
| | leading to the formation of a | | | | |
| | fluid-phase C3 convertase | | | | |
| | [C3(H₂O),Bb]. This fluid-phase | | | | |
| | C3 convertase cleaves C3 from | | | | |
| | serum to produce metastable | | | | |
| | C3b, which binds randomly from | | | | |
| | the fluid phase onto particles. | | | | |
| | Binding of C3 fragments to | | | | |
| | cellular targets opsonizes the | | | | |
| | target cells for efficient | | | | |
| | phagocytosis by cells with | | | | |
| | receptors for C3 fragments. | | | | |
| Complement | The cleavage of C5 is the last | Dry AMD | RPE/Choroid, Glial | RPE/Choroid, Glial | RPE/Choroid, |
| C5 | enzymatic step in the | | cells | cells | Glial cells |
| | complement activation cascade | | | | |
| | resulting in the formation of two | | | | |
| | biologically important fragments, | | | | |
| | C5a and C5b | | | | |
| Complement | cleavage product of C5. C5a is a | Dry AMD | | | |
| C5a | potent chemotactic and | | | | |
| | spasmogenic anaphylatoxin. It | | | | |
| | mediates inflammatory | | | | |
| | responses by stimulating | | | | |
| | neutrophils and phagocytes | | | | |
| Complement | C5b initiates the formation of the | Dry AMD | | | |
| C5b | membrane attack complex (C5b- | | | | |
| | 9), which results in the lysis of | | | | |
| | bacteria, cells and other | | | | |
| | pathogens | | | | |
| Complement | Inhibitor of the complement | Dry AMD | Drusen deposits | Drusen deposits | |
| Factor H | activation pathway. Large | | | | |
| | percentage of people with AMD | | | | |
| | have a SNP in CFH resulting in | | | | |
| | complement pathway activation. | | | | |
| VEGF | VEGF stimulates angiogenesis | Wet AMD | endothelial cells | endothelial cells | endothelial |
| | by being an endothelial cell | | | | cells |
| | mitogen and sustaining | | | | |
| | endothelial cell survival by | | | | |
| | inhibiting apoptosis. VEGF is a | | | | |
| | chemoattractant for endothelial | | | | |
| | cell precursors and promoting | | | | |
| | their differentiation. VEGF is an | | | | |
| | agonist of vascular permeability. | | | | |
| VEGF | VEGF receptor inhibitors block | Wet AMD | endothelial cells | endothelial cells | endothelial |
| receptors (1, | VEGF signaling | | | | cells |
| 2 or both) | | | | | |
| Integrin αᵥ β₃ | upregulated during endothelial | AMD | endothelial cells | endothelial cells | |
| | proliferation during angiogenesis | | | | |
| | and vascular remodeling, | | | | |
| | Involved in VEGF-VEGFr2 | | | | |
| | signaling pathway | | | | |
| PDGF | Involved in angiogenic sprouting | Wet AMD | endothelial cells, | endothelial cells, | endothelial |
| receptor β | of endothelial cells, capillary | | pericytes, smooth | pericytes, smooth | cells, |
| | maturation through pericyte | | muscle cells | muscle cells | pericytes, |
| | recruitment, pericyte viability and | | | | smooth |
| | survival as well as induction of | | | | muscle cells |
| | VEGF signaling in endothelial | | | | |
| | cells. | | | | |
| Protein | PKC is a family of | Wet AMD/ | endothelial cells | endothelial cells | endothelial |
| Kinase C | serine/threonine kinases | Diabetic | | | cells |
| | involved in signal transduction | retinopathy | | | |
| | resulting in cell proliferation, | | | | |
| | differentiation, apoptosis and | | | | |
| | angiogenesis. | | | | |
| c-JUN | Transcription factor involved in | Wet AMD/ | epithelial and | epithelial and | epithelial and |
| transcription | the regulation of genes involved | Diabetic | endothelial cells | endothelial cells | endothelial |
| factor | in endothelial proliferation and | retinopathy | | | cells |
| | neovascularization including | | | | |
| | MMP-2 | | | | |
| IL-1alpha | Inflammatory cytokine produced | Dry Eye | cornea, conj, | Expression is | Increased |
| | by immune cells and the ocular | | choroid, retina | increased in a dry | mRNA under |
| | surface epithelium. Increased IL- | | | eye model in | hyperosmolar |
| | 1alpha is found in tears of dry | | | cornea and conj | and |
| | eye patients and contributes to | | | epithelium | desiccating |
| | immune response during dry eye | | | | conditions |
| IL-1beta | Inflammatory cytokine produced | Dry Eye | cornea, conj, | Expression is | Low basal |
| | by immune cells and the ocular | | choroid, retina | increased in an | expression. |
| | surface epithelium - Some | | | experimental dry | Increased |
| | controversy as to presence and | | | eye model in | expression in |
| | increased amounts in tears | | | cornea and conj | corneal |
| | correlating with dry eye | | | epithelium | epithelium |
| | | | | | when treated |
| | | | | | with estrogen |
| | | | | | (inflammation |
| | | | | | of the eye) |
| TNFalpha | Inflammatory cytokine produced | Dry Eye | cornea, conj, iris, | Expression is | Hyperosmolarity |
| | by macrophages and other | | choroid, retina | increased in an | induces |
| | immune cells present in tears of | | | experimental dry | increased |
| | dry eye patients. Increased | | | eye model in | TNF-alpha |
| | TNF-alpha secreted by the | | | cornea and conj | mRNA in |
| | corneal and conjunctival | | | epithelium | corneal and |
| | contribute to the inflammatory | | | | conj |
| | cascade in dry eye | | | | epithelium |
| MMP | Class of endopeptidases that | Dry Eye | found in all ocular | Elevated levels of | Hyperosmolarity |
| | degrade extracellular matrix | | tissues | MMP-2, MMP-7 | induces |
| | proteins and other | | | and MMP-9 are | increased |
| | molecules/receptors. MMPs | | | found in tears of | MMP-9 |
| | secreted by the ocular surface | | | patients with dry | mRNA in |
| | epithelium may disrupt the mucin | | | eye. Desiccating | corneal and |
| | layer in the tear film, leading to | | | stress and | conj |
| | dry eye | | | hyperosmolarity | epithelium |
| | | | | induce expression | |
| | | | | of MMP-2 and | |
| | | | | MMP-9 in corneal | |
| | | | | epithelium | |
| ICAM-1 | Intracellular adhesion molecule | Dry Eye | cornea, conj, iris, | increased in the | increased in |
| | (ICAM) is an integral membrane | | choroid, retina | conj epithelium of | the conj |
| | protein on the surface of | | | dry eye patients, | epithelium of |
| | leukocytes and endothelial cells | | | low basal | dry eye |
| | and its expression is increased | | | expression in | patients |
| | upon cytokine stimulation. | | | normal patients | |
| | Presence on the ocular surface | | | | |
| | recruits immune cells to the | | | | |
| | epithelium and causes an | | | | |
| | enhanced immune response and | | | | |
| | increased inflammation in dry | | | | |
| | eye. | | | | |
| Insulin like | Insulin-like growth factor 1 is a | Diabetic | endothelial cells | endothelial cells | endothelial |
| growth | mitogenic polypeptide with a | retinopathy/ | | | cells |
| factor-1 | molecular structure similar to | AMD | | | |
| | insulin capable of stimulating | | | | |
| | cellular growth, differentiation | | | | |
| | and metabolism. | | | | |
| Insulin like | IGF-I receptor is comprised of | Diabetic | endothelial cells | endothelial cells | endothelial |
| growth | two extra-cellular alpha-subunits, | retinopathy/ | | | cells |
| factor-1 | containing hormone binding | AMD | | | |
| receptor | sites, and two membrane- | | | | |
| | spanning beta-subunits, | | | | |
| | encoding an intracellular tyrosine | | | | |
| | kinase. Hormone binding | | | | |
| | activates the receptor kinase, | | | | |
| | leading to receptor | | | | |
| | autophosphorylation and tyrosine | | | | |
| | phosphorylation of multiple | | | | |
| | substrates, including the IRS and | | | | |
| | Shc proteins. Through these | | | | |
| | initial tyrosine phosphorylation | | | | |
| | reactions, IGF-I signals are | | | | |
| | transduced to intracellular lipid | | | | |
| | and serine/threonine kinases that | | | | |
| | results in cell proliferation, | | | | |
| | modulation of tissue | | | | |
| | differentiation, and protection | | | | |
| | from apoptosis. | | | | |
| growth | Among other activities GH | Diabetic | endothelial cells | endothelial cells | endothelial |
| hormone | signaling stimulates the | retinopathy | | | cells |
| receptor | production and secretion if IGFs | | | | |
| GHr | | | | | |
| Integrins αᵥ | This integrin functions in a | Diabetic | endothelial cells | endothelial cells | |
| β₅ | similar manner to Integrin αv β3 | retinopathy/ | | | |
| | but may be involved in separate | AMD | | | |
| | signaling pathways | | | | |
| TNFα | TNFα alters endothelial cell | Diabetic | Retina/Cornea | Retinal Muller | Retinal Muller |
| | morphology and behavior, | retinopathy/ | | cells/Cornea/Endothelium | cells |
| | promoting angiogenesis and | AMD | | and vessel | |
| | stimulating mesenchymal cells to | | | walls of | |
| | generate extracellular matrix | | | fibrovascular | |
| | proteins. In activating | | | membranes | |
| | endothelium, TNFa upregulates | | | | |
| | the basal levels of expression of | | | | |
| | ICAM-1. | | | | |
| ICAM-1 | Leukocyte binding to the retinal | Diabetic | endothelial cells | endothelial cells | endothelial |
| | vascular endothelium is involved | retinopathy | | | cells |
| | in the pathogenesis of diabetic | | | | |
| | retinopathy, as it results in early | | | | |
| | blood-retinal barrier breakdown, | | | | |
| | capillary nonperfusion, and | | | | |
| | endothelial cell injury and death. | | | | |
| | Leukocyte adhesion to the | | | | |
| | diabetic retinal vasculature is | | | | |
| | mediated in part by intercellular | | | | |
| | adhesion molecule-1 (ICAM-1), | | | | |
| | which is expressed on | | | | |
| | endothelial cells. | | | | |
| MMP-10 | Overexpression leads to | Diabetic | Cornea | Cornea | Cornea |
| | alterations of corneal BM and | retinopathy | | | |
| | laminin binding integrin α₃/β₁ | | | | |
| MMP-2 | elevated expression of MMPs in | Diabetic | retina, endothelial | endothelial cells | retina |
| | the retina facilitates increased | retinopathy/ | cells | | |
| | vascular permeability by a | AMD | | | |
| | mechanism involving proteolytic | | | | |
| | degradation of the tight junction | | | | |
| | protein occludin followed by | | | | |
| | disruption of the overall tight | | | | |
| | junction complex. MMPs are | | | | |
| | needed for the degradation of | | | | |
| | ECM to facilitate the migration of | | | | |
| | proliferating endothelial cells | | | | |
| MMP-9 | elevated expression of MMPs in | Diabetic | retina, endothelial | endothelial cells | retina |
| | the retina facilitates increased | retinopathy/ | cells | | |
| | vascular permeability by a | AMD | | | |
| | mechanism involving proteolytic | | | | |
| | degradation of the tight junction | | | | |
| | protein occludin followed by | | | | |
| | disruption of the overall tight | | | | |
| | junction complex, MMPs are | | | | |
| | needed for the degradation of | | | | |
| | ECM to facilitate the migration of | | | | |
| | proliferating endothelial cells | | | | |

### EQUIVALENTS

While the invention has been described in connection with the specific embodiments thereof, it will be understood that it is capable of further modification. Furthermore, this application is intended to cover any variations, uses, or adaptations of the invention, including such departures from the present disclosure as come within known or customary practice in the art to which the invention pertains, and as fall within the scope of the appended claims. All references cited above are incorporated herein by reference in their entireties.

What is claimed is:

1. A method of delivering an effective amount of siRNA via transscleral iontophoresis into the eye of a subject, comprising:
a) placing a device on the center of the eyeball surface of the subject such that an application surface is formed between the device and the eyeball, wherein the device comprises a reservoir containing an aqueous solution comprising one or more siRNA molecules or formulations thereof, and wherein the device is connected to an electrical generator; and
b) administering the siRNA to the eye of the subject by performing iontophoresis,
thereby delivering the siRNA into the eye.

2. The method of claim 1, wherein the application of the device to the surface of the eyeball is at least partly limited by an outer line concave towards the optical axis of the eyeball, and wherein the outer wall of the device extends from the outer line outwardly with respect to the optical axis.

3. The method of claim 1, wherein the siRNA is between about 15 and about 30 nucleotides in length.

4. The method of claim 1, wherein the siRNA is between about 21 and about 23 nucleotides in length.

5. The method of claim 1, wherein the reservoir contains a therapeutic composition comprising at least one oligonucleotide compound formulated in an aqueous solution suitable for ocular iontophoresis.

6. The method of claim 5, wherein the therapeutic composition comprises at least agent selected from the group consisting of: a buffering agent, an osmotic agent, a permeation enhancer, a chelant, an antioxidant and an antimicrobial preservative.

7. The method of claim 5, wherein the therapeutic composition is lyophilized prior to being reconstituted for iontophoresis application.

8. The method of claim 1, wherein the reservoir contains an siRNA formulation in the form of a nanoparticle.

9. The method of claim 8, wherein the nanoparticle comprises at least agent selected from the group consisting of: a buffering agent, an osmotic agent, a permeation enhancer, a chelant, an antioxidant and an antimicrobial preservative.

10. The method of claim 8, wherein the nanoparticle has a diameter between about 20 nm and about 400 nm.

11. The method of claim 8, wherein the nanoparticle has a hydrodynamic diameter between about 40 nm and about 200 nm.

12. The method of claim 8, wherein the nanoparticle has a zeta potential between about +5 mV and about +100 mV.

13. The method of claim 8, wherein the nanoparticle has a zeta potential between about +20 mV and about +80 mV.

14. The method of claim 8, wherein the nanoparticle has a zeta potential between about −5 mV and about −100 mV.

15. The method of claim 8, wherein the nanoparticle has a zeta potential between about −20 mV and about −80 mV.

16. The method of claim 8, wherein the nanoparticle is delivered by an iontophoretic current between about +0.25 mA and about +10 mA.

17. The method of claim 8, wherein the nanoparticle is delivered by an iontophoretic current between about +0.5 mA and about +5 mA.

18. The method of claim 1, wherein the reservoir holds between about 50 μL to about 500 μL, of the siRNA formulation.

19. The method of claim 1, wherein the reservoir holds between about 150 μL to about 400 μL, of the siRNA formulation.

20. The method of claim 1, wherein the administration time is between about 1 minute and about 20 minutes.

21. The method of claim 1, wherein the administration time is between about 2 minutes and about 10 minutes.

22. The method of claim 1, wherein the administration time is between about 3 minutes and about 5 minutes.

23. The method of claim 1, wherein the siRNA in solution is delivered by an iontophoretic current between about −0.25 mA and about −10 mA.

24. The method of claim 23, wherein the siRNA in solution is delivered by an iontophoretic current between about −0.5 mA and about −5 mA.

25. The method of claim 1, wherein administration of siRNA occurs in a single dose.

26. The method of claim 1, wherein administration of siRNA occurs over multiple doses.

27. The method of claim 1, wherein the oligonucleotide is delivered by injection prior to iontophoresis.

28. The method of claim 27, wherein the method of injection is selected from the group consisting of: an intracameral injection, an intracorneal injection, a subconjunctival injection, a subtenon injection, a subretinal injection, an intravitreal injection and an injection into the anterior chamber.

29. The method of claim 1, wherein the oligonucleotide is administered topically prior to iontophoresis.

30. The method of claim 1, wherein the step of ocular iontophoresis is carried out prior to, during or after the step of administering oligonucleotide.

31. A method for treating ocular diseases in a mammal, comprising administering an effective amount of siRNA by ocular iontophoresis.

32. An siRNA formulation suitable for ocular iontophoretic delivery into the eye of a subject.

33. The siRNA formulation of claim 32, wherein the formulation comprises a nanoparticle composition comprising the siRNA.

34. A device for delivering siRNA to the eye of a subject, comprising:
a) a reservoir comprising at least one medium comprising a siRNA formulation, the reservoir extending along a surface intended to cover a portion of an eyeball; and
b) an electrode associated with the reservoir,
wherein when the reservoir is placed in contact with the eyeball, the electrode can supply an electric field directed through the medium and toward a surface of the eye, thereby causing the siRNA to migrate into the eye and thereby delivering the siRNA formulation through the surface of the eye through iontophoresis.

35. The device of claim 34, wherein the reservoir comprises:
a) a first container for receiving the at least one medium comprising the siRNA formulation;
b) a second container for receiving an electrical conductive medium comprising electrical conductive elements; and
c) a semi-permeable membrane positioned between the first and second containers, the semi-permeable membrane being permeable to electrical conductive elements and non-permeable to the active substances.
